(19) United States
(12) Patent Application Publication
(10) Pub. No.: US 2025/0345495 A1
(43) Pub. Date: Nov. 13, 2025

(54) RED BLOOD CELL ADDITIVE SOLUTION MANAGEMENT AND STORAGE OF RED BLOOD CELL PRODUCTS

(71) Applicant: Fenwal, Inc., Lake Zurich, IL (US)

(72) Inventors: Benjamin E. Kusters, Pleasant Prairie, WI (US); Kyungyoon Min, Kildeer, IL (US)

(21) Appl. No.: 18/869,472

(22) PCT Filed: May 26, 2023
(86) PCT No.: PCT/US2023/067570

§ 371 Date: Nov. 26, 2024

Related U.S. Application Data
(60) Provisional application No. 63/347,382, filed on May 31, 2022.

(51) Int. Cl. A61M 1/02

(52) U.S. Cl. CPC A61M 1/0281; A61M 1/0218; A61M 1/029; A61M 2205/3393

(57) ABSTRACT

A blood processing system includes a durable hardware component and a single-use fluid flow circuit. The hardware component includes a controller configured to execute a procedure in which red blood cells are separated from whole blood and collected. An additive solution may be initially provided in a red blood cell collection container of the fluid flow circuit, with the controller actuating a pump system of the hardware component to convey the additive solution from the red blood cell collection container into an additive solution container of the fluid flow circuit. After red blood cells have been separated from blood and conveyed into the red blood cell collection container (along with at least a portion of the additive solution from the additive solution container), the red blood cells and additive solution in the red blood cell collection container may be conveyed into a whole blood container for collection and storage.

### CROSS-REFERENCE TO RELATED APPLICATIONS

This application claims the benefit of and priority to U.S. Provisional Patent Application Ser. No. 63/347,382, filed May 31, 2022, the content of which is hereby incorporated by reference.

### BACKGROUND

### Field of the Disclosure

The present disclosure relates generally to separation and collection of red blood cells (“RBCs”) from blood. More particularly, the present disclosure relates to apparatus and methods for managing a red blood cell additive solution and/or for storing a red blood cell product using one or more containers omitting di-2-ethylhexyl phthalate (“DEHP”).

### Description of Related Art

Blood and blood components are widely used in medical applications. Typically, whole blood collected from a donor is processed further. Often, the blood is processed to obtain blood components such as RBCs, plasma, and platelets. This can, for example, be done by collection of whole blood, followed by filtration and subsequently by centrifugation, by collection of whole blood, followed by centrifugation and subsequently by filtration, or by the automated collection of components.

RBCs are often separated from collected whole blood and transfused later to a patient in need thereof. For example, RBCs may be administered to a patient suffering from a loss of blood due to trauma, as a post-chemotherapy treatment, or as part of a treatment for one or more blood-borne diseases. Unless administered immediately after collection and separation, RBCs are typically stored for some period of time prior to transfusion. The storage period may vary from a few days to several weeks. It is typical for plasticized polyvinylchloride (“PVC”)-based materials

and solutions to be used for collecting, processing, storing, and transfusing blood and blood components. Due to its properties, PVC is highly preferred for these applications, in particular for the use in transfusion systems. However, PVC is rather brittle and therefore is used along with a plasticizer or extractable agent to ensure the required flexibility and softness of the material. Thus typically, ortho-phthalates (hereinafter also designated as “phthalates”) such as DEHP are used as plasticizer or extractable agents for PVC.

While DEHP is an effective plasticizer or extractable agent for PVC, leaching of DEHP from a container of an extracorporeal fluid flow circuit to a (biological or non-biological) fluid stored within the container is possible. DEHP has been found to improve RBC quality during storage (by reducing hemolysis), but certain recipients of blood or blood components are considered particularly sensitive to DEHP (and possible adverse health effects), such as pregnant women and neonates, because of the greater potential for interaction. Thus, while the leaching of the DEHP plasticizer from the used materials on the one hand may have a positive impact on the quality of blood components, there is an increasing need to provide blood and blood components that are essentially DEHP-free (or more preferably essentially phthalate-free) to individuals in need thereof.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a blood processing system includes a reusable processing device and a disposable fluid flow circuit. The processing device has a pump system, a blood separation assembly, and a controller configured to execute a blood processing procedure. The disposable fluid flow circuit has a processing chamber received by the blood separation assembly, a red blood cell collection container containing an additive solution, an additive solution container, and a plurality of conduits fluidly connecting the components of the fluid flow circuit. The controller is configured to actuate the pump system to convey the additive solution from the red blood cell collection container into the additive solution container, actuate the pump system to convey blood from a blood source into the processing chamber, and actuate the blood separation assembly to separate red blood cells from the blood in the processing chamber. The controller then actuates the pump system to convey at least a portion of the separated red blood cells out of the processing chamber and into the red blood cell collection container, and actuates the pump system to convey at least a portion of the additive solution from the additive solution container into the red blood cell collection container.

In another aspect, a method is provided for separating red blood cells from whole blood. The method includes conveying an additive solution from a red blood cell collection container into an additive solution container, conveying blood from a blood source into a processing chamber, and then separating red blood cells from the blood in the processing chamber. At least a portion of the separated red blood cells is conveyed out of the processing chamber and into the red blood cell collection container, with at least a portion of the additive solution being conveyed from the additive solution container into the red blood cell collection container.

In yet another aspect, a blood processing system includes a reusable processing device and a disposable fluid flow circuit. The processing device has a pump system, a blood separation assembly, and a controller configured to execute a blood processing procedure. The fluid flow circuit has a processing chamber received by the blood separation assembly, a red blood cell collection container, a whole blood container, and a plurality of conduits fluidly connecting the components of the fluid flow circuit. The controller is configured to actuate the pump system to convey blood from the whole blood container into the processing chamber and then actuate the blood separation assembly to separate red blood cells from the blood in the processing chamber. The controller next actuates the pump system to convey at least a portion of the separated red blood cells out of the processing chamber and into the red blood cell collection container and then actuates the pump system to convey said at least a portion of the separated red blood cells from the red blood cell collection container into the whole blood container.

In another aspect, a method is provided for separating red blood cells from whole blood. The method includes conveying blood from a whole blood container into a processing chamber and then separating red blood cells from the blood in the processing chamber. Next, at least a portion of the separated red blood cells is conveyed out of the processing chamber and into a red blood cell collection container, and then said at least a portion of the separated red blood cells is conveyed out of the red blood cell collection container and into the whole blood container.

These and other aspects of the present subject matter are set forth in the following detailed description of the accompanying drawings.

### BRIEF DESCRIPTION OF THE DRAWINGS

FIG. 1 is a perspective view of an exemplary reusable hardware component of a blood processing system which is configured to receive a disposable fluid flow circuit;

FIG. 2 is a plan view of an exemplary disposable fluid flow circuit for use in combination with the durable hardware component of FIG. 1;

FIG. 3 is a schematic view of the fluid flow circuit of FIG. 2 mounted to the processing device of FIG. 1 to complete a blood processing system according to an aspect of the present disclosure;

FIG. 4 is a schematic view of the blood processing system of FIG. 3 executing an “additive solution transfer” stage of an exemplary blood processing procedure;

FIG. 5 is a schematic view of the blood processing system of FIG. 3 executing a “blood prime” stage of an exemplary blood processing procedure;

FIG. 6 is a schematic view of the blood processing system of FIG. 3 executing an “establish separation” stage of an exemplary blood processing procedure;

FIG. 7 is a schematic view of the blood processing system of FIG. 3 executing a “collection” stage of an exemplary blood processing procedure, with separated RBCs being leukoreduced before collection;

FIG. 8 is a schematic view of a variation of the “collection” stage of FIG. 7 in which the separated RBCs are not leukoreduced before collection;

FIG. 9 is a schematic view of the blood processing system of FIG. 3 executing a “red blood cell recovery” stage of an exemplary blood processing procedure, with separated RBCs being leukoreduced before collection;

FIG. 10 is a schematic view of a variation of the “red blood cell recovery” stage of FIG. 9 in which the separated RBCs are not leukoreduced before collection;

FIG. 11 is a schematic view of the blood processing system of FIG. 3 executing an “additive solution flush” stage of an exemplary blood processing procedure, with additive solution being directed through a leukoreduction filter before entering a red blood cell collection container;

FIG. 12 is a schematic view of a variation of the “additive solution flush” stage of FIG. 11 in which the additive solution enters the red blood cell collection container without passing through the leukoreduction filter;

FIG. 13 is a schematic view of the blood processing system of FIG. 3 executing “red blood cell transfer” and “air evacuation” stages of an exemplary blood processing procedure; and

FIG. 14 is a schematic view of the blood processing system of FIG. 3 executing a “sealing” stage of an exemplary blood processing procedure.

### DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific designs and features disclosed herein are not to be interpreted as limiting the subject matter as defined in the accompanying claims.

The embodiments disclosed herein are for the purpose of providing an exemplary description of the present subject matter. They are, however, only exemplary and not exclusive, and the present subject matter may be embodied in various forms. Therefore, specific details disclosed herein are not to be interpreted as limiting the subject matter as defined in the accompanying claims.

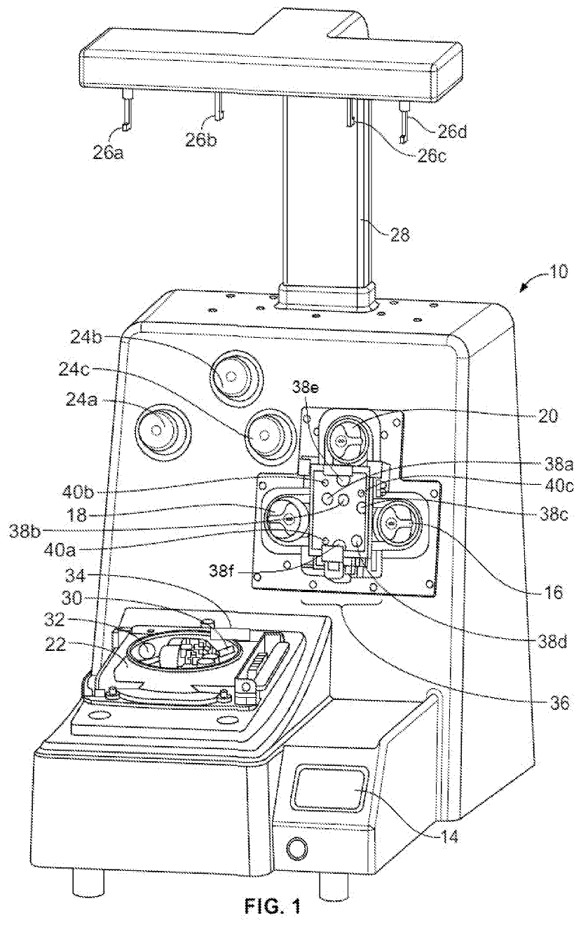

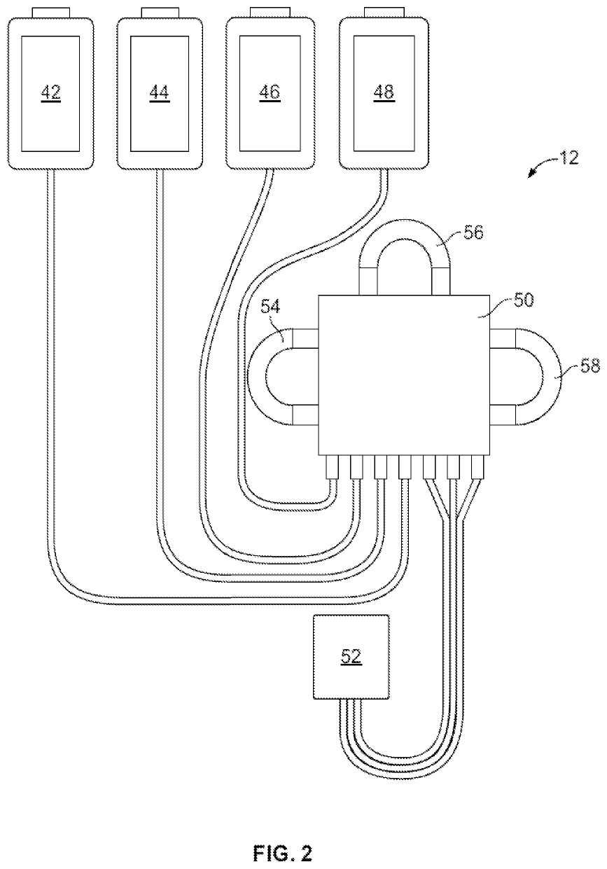

FIG. 1 depicts a reusable or durable hardware component or processing device of a configurable, automated blood processing system or blood component manufacturing system, generally designated 10, while FIG. 2 depicts a disposable or single-use fluid flow circuit, generally designated 12, to be used in combination with the processing device 10 for processing collected whole blood. The illustrated processing device 10 and fluid flow circuit 12 are generally configured as described in PCT Patent Application Publication No. WO 2021/194824 A1 (which is hereby incorporated herein by reference), but it should be understood that the processing device 10 and associated fluid flow circuit 12 may be differently configured without departing from the scope of the present disclosure.

The illustrated processing device 10 includes associated pumps, valves, sensors, displays, and other apparatus for configuring and controlling flow of fluid through the fluid flow circuit 12, described in greater detail below. The blood processing system may be directed by a controller integral with the processing device 10 that includes a programmable microprocessor to automatically control the operation of the pumps, valves, sensors, etc. The processing device 10 may also include wireless communication capabilities to enable the transfer of data from the processing device 10 to the quality management systems of the operator.

More specifically, the illustrated processing device 10 includes a user input and output touchscreen 14, a pump station or system including a first pump 16 (for pumping, e.g., whole blood), a second pump 18 (for pumping, e.g., plasma) and a third pump 20 (for pumping, e.g., additive solution), a centrifuge mounting station and drive unit 22 (which may be referred to herein as a “centrifuge”), and clamps 24a-c. While blood separation will be described herein as being achieved via centrifugation, it should be understood that the present disclosure is not limited to blood separation via centrifugation, but rather encompasses any suitable approach and blood separation assembly for separating blood into two or more components.

For example, in one embodiment, the centrifuge may be replaced with a spinning membrane-type blood separation assembly of the type described in U.S. Patent Application Publication No. 2019/0201916, which is hereby incorporated herein by reference.

The touchscreen 14 enables user interaction with the processing device 10, as well as the monitoring of procedure parameters, such as flow rates, container weights, pressures, etc. The pumps 16, 18, and 20 (collectively referred to herein as being part of a “pump system” of the processing device 10) are illustrated as peristaltic pumps capable of receiving tubing or conduits and moving fluid at various rates through the associated conduit dependent upon the procedure being performed. An exemplary centrifuge mounting station/drive unit is seen in U.S. Pat. No. 8,075,468 (with reference to FIGS. 26-28), which is hereby incorporated herein by reference. The clamps 24a-c (collectively referred to herein as being part of the “valve system” of the processing device 10) are capable of opening and closing fluid paths through the tubing or conduits and may incorporate RF sealers in order to complete a heat seal of the tubing or conduit placed in the clamp to seal the tubing or conduit leading to a product container upon completion of a procedure. Sterile connection/docking devices may also be incorporated into one

or more of the clamps 24a-c. The sterile connection devices may employ any of several different operating principles. For example, known sterile connection devices and systems include radiant energy systems that melt facing membranes of fluid flow conduits, as in U.S. Patent No. 4,157,723; heated wafer systems that employ wafers for cutting and heat bonding or splicing tubing segments together while the ends remain molten or semi-molten, such as in U.S. Pat. Nos. 4,753,697; 5,158,630; and 5,156,701; and systems employing removable closure films or webs sealed to the ends of tubing segments as described, for example, in U.S. Pat. No. 10,307,582. Alternatively, sterile connections may be formed by compressing or pinching a sealed tubing segment, heating and severing the sealed end, and joining the tubing to a similarly treated tubing segment as in, for example, U.S. Pat. Nos. 10,040,247 and 9,440,396. All of the above-identified patents are incorporated by reference in their entirety. Sterile connection devices based on other operating principles may also be employed without departing from the scope of the present disclosure.

The processing device 10 also includes hangers 26a-d (which may each be associated with a weight scale) for suspending the various containers of the disposable fluid circuit 12. The hangers 26a-d are preferably mounted to a support 28, which is vertically translatable to improve the transportability of the processing device 10. An optical system comprising a laser 30 and a photodetector 32 is associated with the centrifuge 22 for determining and controlling the location of an interface between separated blood components within the centrifuge 22. An exemplary optical system is shown in U.S. Patent Application Publication No. 2019/0201916. An optical sensor 34 is also provided to optically monitor one or more conduits leading into or out of the centrifuge 22.

The face of the processing device 10 includes a nesting module 36 for seating a flow control cassette 50 (FIG. 2) of the fluid flow circuit 12 (described in greater detail below). The cassette nesting module 36 is configured to receive various disposable cassette designs so that the system may be used to perform different types of procedures. Embedded within the illustrated cassette nesting module 36 are four valves 38a-f (collectively referred to herein as being part of the “valve system” of the processing device 10) for opening and closing fluid flow paths within the flow control cassette 50, and three pressure sensors 40a-c capable of measuring the pressure at various locations of the fluid flow circuit 12.

With reference to FIG. 2, the illustrated fluid flow circuit 12 includes a plurality of containers 42, 44, 46, and 48, with a flow control cassette 50 and a processing/separation chamber 52 that is configured to be received in the centrifuge 22, all of which are interconnected by conduits or tubing segments, so as to permit continuous flow centrifugation. The flow control cassette 50 routes the fluid flow through three tubing loops 54, 56, 58, with each loop being positioned to engage a particular one of the pumps 16, 18, 20. The conduits or tubing may extend through the cassette 50, or the cassette 50 may have pre-formed fluid flow paths that direct the fluid flow.

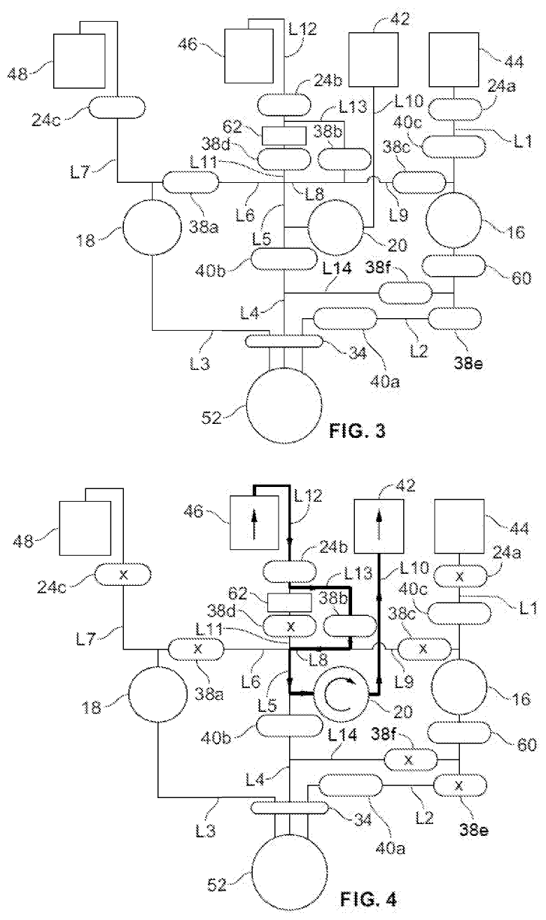

In the fluid flow circuit 12 shown in FIG. 2, container 42 may be pre-filled with additive solution, container 44 may be filled with whole blood and connected to the fluid flow circuit 12 at the time of use, container 46 may be an empty container for the receipt of RBCs separated from the whole blood, and container 48 may be an empty container for the receipt of plasma separated from the whole blood. While FIG. 2 shows a whole blood container 44 (configured as a blood pack unit, for example) as a blood source, it is within the scope of the present disclosure for the blood source to be a living donor, as will be described in greater detail herein. Additionally, while container 42 may be pre-filled with additive solution, it is also within the scope of the disclosure for container 42 to be empty and for one of the other containers to be pre-filled with additive solution, with all or a portion of the additive solution being conveyed into container 42 during a blood processing procedure (as will be described in greater detail herein). The fluid flow circuit may optionally include an air trap 60 (FIG. 3) through which the whole blood is flowed prior to entering the processing chamber 52 and/or a leukoreduction filter 62 through which the RBCs are flowed prior to entering the red blood cell collection container 46.

The processing chamber 52 may be pre-formed in a desired shape and configuration by injection molding from a rigid plastic material, as shown and described in U.S. Pat. No. 6,849,039, which is hereby incorporated herein by reference. The specific geometry of the processing chamber 52 may vary depending on the elements to be separated, and the present disclosure is not limited to the use of any specific chamber design. For example, it is within the scope of the present disclosure for the processing chamber 52 to be configured formed of a generally flexible material, rather than a generally rigid material. When the processing chamber 52 is formed of a generally flexible material, it relies upon the centrifuge 22 to define a shape of the processing chamber 52. An exemplary processing chamber formed of a flexible material and an associated centrifuge are described in U.S. Pat. No. 6,899,666, which is hereby incorporated herein by reference.

In an exemplary embodiment, the controller of the processing device 10 is pre-programmed to automatically operate the system to perform one or more standard blood processing procedures selected by an operator by input to the touchscreen 14, and configured to be further programmed by the operator to perform additional blood processing procedures. The controller may be pre-programmed to substantially automate a wide variety of procedures, including, but not limited to: RBC and plasma production from a single unit of whole blood (as will be described in greater detail herein), buffy coat pooling, buffy coat separation into a platelet product (as described in U.S. Patent Application Publication No. 2018/0078582, which is hereby incorporated herein by reference), glycerol addition to RBCs, RBC washing, platelet washing, and cryoprecipitate pooling and separation.

The pre-programmed blood processing procedures operate the system at pre-set settings for flow rates and centrifugation forces, and the programmable controller may be further configured to receive input from the operator as to one or more of flow rates and centrifugation forces for the standard blood processing procedure to override the pre-programmed settings.

In addition, the programmable controller is configured to receive input from the operator through the touchscreen 14 for operating the system to perform a non-standard blood processing procedure. More particularly, the programmable controller may be configured to receive input for settings for the non-standard blood processing procedure, including flow rates and centrifugation forces.

In an exemplary procedure, the processing device 10 and the fluid flow circuit 12 may be used in combination to process an amount of whole blood (e.g., one unit) into a red blood cell product and a plasma product. FIG. 3 is a schematic illustration of the fluid flow circuit 12 mounted to the processing device 10, with selected components of the fluid flow circuit 12 and selected components of the processing device 10 being shown. FIGS. 4-14 show different stages of an exemplary blood processing procedure.

As described above, it is within the scope of the present disclosure for the additive solution container 42 to either be pre-filled with an additive solution or empty, with some other container being pre-filled with the additive solution. For example, in the illustrated embodiment, the additive solution container 42 is empty (or at least substantially empty), with the red blood cell collection container 46 being pre-filled with an additive solution (e.g., ADSOL®). An advantage of such a configuration is that it allows for the use of a non-DEHP plasticized container (namely, the red blood cell collection container 46) for the storage of RBCs produced during a blood separation procedure, while the additive solution container 42 may be formed of a material including DEHP. Such an approach reduces the time and cost of executing a blood separation procedure compared to the conventional approach. More particularly, it is conventional for an additive solution to be

contained within a dedicated additive solution container. A container filled with additive solution cannot undergo radiation sterilization, such that the additive solution container must be provided separately from the remainder of the associated fluid flow circuit (which is sterilized via radiation) and separately sterilized (e.g., using steam). Once the additive solution container has been sterilized, it is sterilely connected to one of the conduits of the fluid flow circuit, just prior to use of the fluid flow circuit.

Similar to a container filled with an additive solution, a container formed of a material omitting DEHP (e.g., a citrate-plasticized material) is also incompatible with radiation sterilization and, thus, also requires sterile connection to the associated fluid flow circuit after sterilization. Accordingly, when a fluid flow circuit is provided with a non-DEHP red blood cell collection container and an additive solution container pre-filled with an additive solution, sterile connection of the two containers to the remainder of the fluid flow circuit is required, which increases the time and cost of any blood separation procedure. On the other hand, if the additive solution were initially provided in a non-DEHP red blood cell collection container 46, only one sterile connection (of the non-DEHP red blood cell collection container 46) is required, as the empty additive solution container 42 (which may be formed of a material including DEHP) may be pre-attached to the fluid flow circuit 12 and radiation sterilized therewith. As should be clear, this reduces the time and cost of executing a blood separation procedure.

Once the additive solution-filled red blood cell collection container 46 (which may be formed of a citrate-plasticized material or any other suitable non-DEHP material) has been attached to the remainder of the fluid flow circuit 12, an initial step of a blood processing procedure may be executed to convey the additive solution from the red blood cell collection container 46 into the additive solution container 42. When this “additive solution transfer” stage (which is shown in FIG. 4) has been completed, the blood processing procedure may proceed as usual.

During the additive solution transfer stage, additive solution is drawn from the red blood cell collection container 46 via line L12 by operation of the third pump 20 (which may be referred to as the “additive pump”). Clamp 24b and valve 38b are open, while the other clamps and valves are closed, which directs the additive solution from line L12 into and through line L13, then into and through line L8, then into and through line L5, and then through line L10 and into the additive solution container 42. It should be understood that, in FIGS. 4-14, arrows on the containers represent the direction of fluid flow between the container and the conduit connected to the container. For example, line L12 is shown as being connected to the top of the red blood cell collection container 46, such that an upward arrow (as in FIG. 4) represents upward fluid flow out of the red blood cell collection container 46. In contrast, line L10 is shown as being connected to the bottom of the additive solution container 42, such that an upward arrow (as in FIG. 4) represents upward fluid flow into the additive solution container 42.

While FIG. 4 shows the additive solution being routed around the leukoreduction filter 62, it is within the scope of the present disclosure for the additive solution to instead be pumped through the leukoreduction filter 62 (by closing valve 38b and opening valve 38d). In either case, the additive solution transfer stage may continue until all or a particular amount of the additive solution has been transferred from the red blood cell collection container 46 into the additive solution container 42, which may be determined by measuring the weight of either or both containers 42 and 46, by monitoring the operation of the additive pump 20 (to ensure that a proper volume of fluid has been conveyed out of the red blood cell collection container 46), or by any other suitable approach.

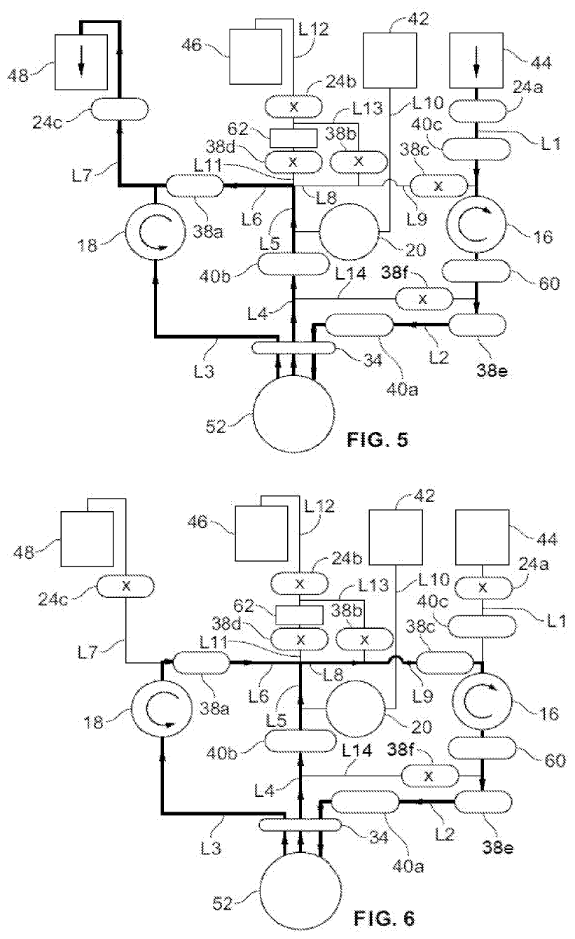

Once the additive solution is in the additive solution container 42 (whether initially provided therein or conveyed therein during an additive solution transfer stage), the procedure may continue with a stage which is referred to herein as a “blood prime” stage. In such a stage (which is shown in FIG. 5), selected components of the fluid flow circuit 12 are primed using blood from a blood source. The blood source is shown in FIG. 5 as the whole blood container 44, but may alternatively be a living donor. Thus, it should be understood that the term “whole blood” may refer to blood that either includes or omits an anticoagulant fluid.

During the blood prime stage, whole blood is drawn into the fluid flow circuit 12 from the blood source (the whole blood container 44 in the embodiment of FIG. 5) via line L1 by operation of the first pump 16 (which may be referred to as the “whole blood pump”). Valves 38c and 38f are closed, which directs the blood through pressure sensor 40c and into and through line L2. The blood passes through air trap 60, pressure sensor 40a (which measures the pressure of the processing chamber 52), and optical sensor 34 before flowing into the processing chamber 52, which is positioned within the centrifuge 22 of the processing device 10.

The centrifuge 22 may be stationary during the blood prime stage or may instead be controlled by the controller of the processing device 10 to spin at a low rotation rate (e.g., on the order of approximately 1,000-2,000 rpm). It may be advantageous for the centrifuge 22 to rotate during the blood prime stage in order to create enough g-force to ensure that the air in the processing chamber 52 (which includes air already present in the processing chamber 52, along with air moved into the processing chamber 52 from lines L1 and/or L2 by the flow of blood) is forced towards the low-g (radially inner) wall of the processing chamber 52. Higher centrifuge rotation rates, such as 4,500 rpm (which is required for steady state separation, as will be described) may be undesirable as air blocks (in which air gets stuck and cannot be forced out of the processing chamber 52, causing pressure to rise) are more likely at higher g-forces.

The blood entering the processing chamber 52 will move towards the high-g (radially outer) wall of the processing chamber 52, displacing air towards the low-g wall. A plasma outlet port of the processing chamber 52 is associated with the low-g wall of the processing chamber 52, such that most of the air will exit the processing chamber 52 via the plasma outlet port and associated line L3, although some air may also exit the processing chamber 52 via a red blood cell outlet port associated with the high-g wall of the processing chamber 52.

Valves 38b and 38d are closed, while the second pump 18 (which may be referred to as the “plasma pump”) is active and the additive pump 20 is inactive. Such an arrangement will direct the air exiting the processing chamber 52 via the red blood cell outlet port through associated line L4 and pressure sensor 40b, into line L5 and then into line L6. Valve 38a is open, such that the air flowing through line L6 will meet up with the air flowing through line L3 (i.e., the air that exits the processing chamber 52 via the plasma outlet port). The combined air will flow through line L7 and open clamp 24c, into the plasma collection container 48.

The flow of air out of the processing chamber 52 via either outlet port is monitored by the optical sensor 34, which is capable of determining the optical density of the fluid flowing through the monitored lines and discerning between air and a non-air fluid in lines L3 and L4. When a non-air fluid is detected in both lines L3 and L4, the controller of the processing device 10 will end the blood prime stage and move on to the next stage of the procedure. The amount of blood drawn into the fluid flow circuit 12 from the blood source during the blood prime stage will vary depending on a number of factors (e.g., the amount of air in the fluid flow circuit 12), but may be on the order of approximately 50 to 100 mL. The blood prime stage may take on the order of one to two minutes.

While FIG. 5 illustrates the fluid flow circuit 12 being primed using blood, it should be understood that the fluid flow circuit 12 may be primed (as necessary) with a separately provided fluid (e.g., anticoagulant or saline).

The next stage (shown in FIG. 6) is referred to herein as the “establish separation” stage. Once non-air fluid has been detected in lines L3 and L4, the rotational speed of the centrifuge 22 will be increased to a rate that is sufficient to separate blood into packed RBCs and platelet-poor plasma (which may be in the range of approximately 4,500 to 5,500 rpm, for example). To produce a plasma product that is low in platelets, it may be advantageous for the processing chamber 52 to be configured with a plasma outlet port that is spaced from and positioned downstream of the blood inlet port, rather than being positioned adjacent to the blood inlet port. Such a configuration allows the platelets to settle down into a distinct layer between the plasma and the RBCs (commonly referred to as a “buffy coat”) before the plasma is removed from the processing chamber 52, thus allowing the separated plasma to be platelet-depleted. As for the whole blood pump 16, it continues to operate, but no additional blood is drawn into the fluid flow circuit 12 from the blood source during the establish separation stage (as will be described).

In cases where the blood source includes (in the case of a whole blood container) or provides (in the case of a living donor) only a limited amount of whole blood (e.g., a single unit), the system must work with a finite fluid volume. To avoid product loss or quality issues, the plasma and RBCs initially separated from the blood in the processing chamber 52 and removed from the processing chamber 52 are not directed to their respective collection containers, but are instead mixed together to form recombined whole blood and recirculated back into the processing chamber 52.

More particularly, during the establish separation stage, separated plasma will exit the processing chamber 52 via the plasma outlet port and associated line L3. Clamp 24c is closed during this stage, while valve 38a remains open, which directs the plasma from line L3 into line L6. Separated RBCs exit the processing chamber 52 via the red blood cell outlet port and associated line L4. In the illustrated embodiment, there is no pump associated with line L4, such that the RBCs exit the processing chamber 52 at a rate that is equal to the difference between the rate of the whole blood pump 16 and the rate of the plasma pump 18. In alternative embodiments, there may be a pump associated with the red blood cell outlet line instead of the plasma outlet line or a first pump associated with the plasma outlet line and a second pump associated with the red blood cell outlet line.

The additive pump 20 is inactive during this stage, thereby directing the RBCs from line L4 into line L5. The plasma flowing through line L6 is mixed with the RBCs flowing through line L5 at a junction of the two lines L5 and L6 to form recombined whole blood. Valve 38d is closed, which directs the recombined whole blood into line L8. Valve 38b is also closed, which directs the recombined whole blood from line L8 into line L9 and through open valve 38c. With clamp 24a being closed, the whole blood pump 16 draws the recombined whole blood into line L2 from line L9 (rather than drawing additional blood into the fluid flow circuit 12 from the blood source), with the recombined blood passing through air trap 60, pressure sensor 40a, and optical sensor 34 before flowing back into the processing chamber 52, where it is again separated into plasma and RBCs.

The establish separation stage continues until steady state separation has been achieved, which may take on the order of approximately one to two minutes. As used herein, the phrase “steady state separation” refers to a state in which blood is separated into its constituents in the processing chamber 52, with the radial position of the interface between separated components within the processing chamber 52 being at least substantially maintained (rather than moving radially inwardly or outwardly). The position of the interface may be determined and controlled according to any suitable approach, including using an interface detector of the type described in U.S. Patent Application Publication No. 2019/0201916.

Preferably, steady state separation is achieved with the interface between separated components within the processing chamber 52 at a target location. The target location may correspond to the location of the interface at which separation efficiency is optimized, with the precise location varying depending on a number of factors (e.g., the hematocrit of the whole blood). However, in an exemplary embodiment, the target location of the interface may be the position of the interface when approximately 52% of the thickness or width (in a radial direction) of the channel defined by the processing chamber 52 is occupied by RBCs. In the illustrated embodiment, the position of the interface within the processing chamber 52 may be adjusted by changing the flow rate of the plasma pump 18, with the flow rate being increased to draw more separated plasma out of the processing chamber 52 (which decreases the thickness of the plasma layer within the processing chamber 52) and move the interface toward the low-g wall or decreased to draw less plasma out of the processing chamber 52 (which increases the thickness of the plasma layer within the processing chamber 52) and move the interface toward the high-g wall.

In an exemplary procedure, the controller of the processing device 10 will control the whole blood pump 16 to operate at a constant rate, with the plasma pump 18 initially operating at the same rate, which will quickly increase the thickness of the RBC layer within the processing chamber 52 and move the interface toward the low-g wall. The rate of the plasma pump 18 is gradually decreased as the thickness of the RBC layer increases and the location of the interface approaches the target location. As described above, the target location of the interface may depend upon the hematocrit of the whole blood, meaning that the rate of the plasma pump 18 (which controls the position of the interface) may also depend on the hematocrit of the whole blood. In one embodiment, this relationship may be expressed as follows:

Theoretical plasma pump rate=whole blood pump rate−((whole blood hematocrit*whole blood pump rate)/hematocrit of separated RBCs) [Equation 1]

The hematocrit of the whole blood may be measured before the procedure begins or by the optical sensor 34 during the procedure, while the hematocrit of the separated RBCs may be determined during the procedure by the optical sensor 34 monitoring line L4. In practice, the plasma pump rate will typically not remain at the theoretical rate once steady state separation has been achieved, with the interface at the target location, but rather the plasma pump rate will instead tend to “flutter” around the theoretical rate.

Regardless of the particular manner in which the controller of the processing device 10 executes the establish separation stage and arrives at steady state separation, once steady state separation has been established, the controller ends the establish separation stage and advances the procedure to a “collection” stage, which is illustrated in FIG. 7. At the beginning of the collection stage, the centrifuge 22, the whole blood pump 16, and the plasma pump 18 all continue operating at the same rates at which they were operating at the end of the establish separation stage. The valve system of the processing device 10, however, is adjusted to direct the separated plasma and RBCs to their respective collection containers (rather than recombining them and recirculating them through the centrifuge 22), while causing additional blood to be drawn into the fluid flow circuit 12 from the blood source until a target amount of whole blood (e.g., one unit) has been drawn into the fluid flow circuit 12.

More particularly, during the collection stage, valve 38c is closed and clamp 24a is opened, which causes the whole blood pump 16 to draw additional blood into line L1 from the blood source (which is the whole blood container 44 in the illustrated embodiment, but may be a living donor). The whole blood pump 16 draws the blood from the blood source into line L2 from line L1, with the blood passing through air trap 60, pressure sensor 40a, and optical sensor 34 before flowing into the processing chamber 52, where it is separated into plasma and RBCs. Most of the platelets of the whole blood will remain in the processing chamber 52, along with some white blood cell populations (much as mononuclear cells), while larger white blood cells, such as granulocytes, may exit with the packed RBCs.

The separated plasma exits the processing chamber 52 via the plasma outlet port and associated line L3. Valve 38a is closed, which directs the plasma from line L3 into line L7, through open clamp 24c, and into the plasma collection container 48.

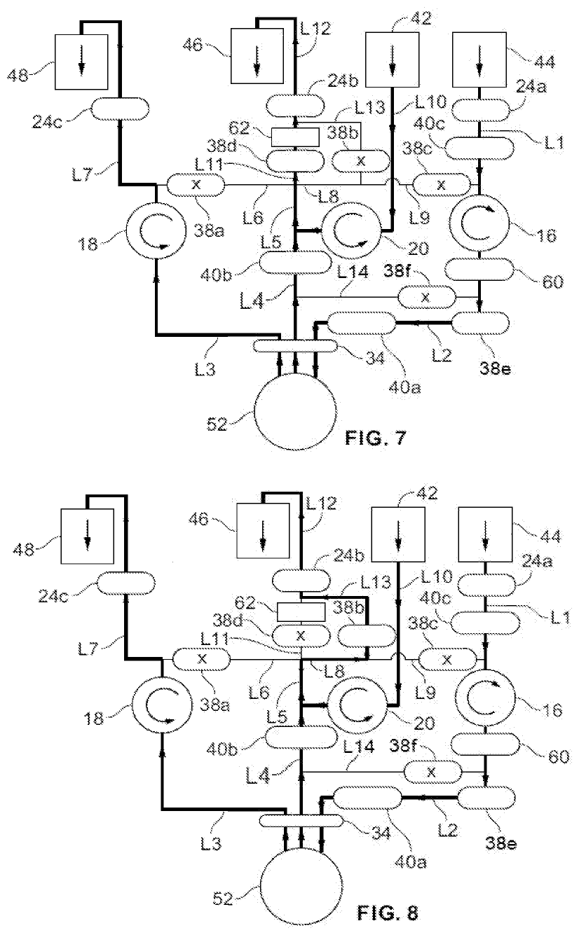

As for the separated RBCs, they exit the processing chamber 52 via the red blood cell outlet port and associated line L4. The additive pump 20 is operated by the controller to draw the additive solution from the additive solution container 42 via line L10. The RBCs flowing through line L4 are mixed with the additive solution flowing through line L10 at a junction of the two lines L4 and L10 to form a mixture that continues flowing into and through line L5. The mixture is ultimately directed into the red blood cell collection container 46, but may first be conveyed through the leukoreduction filter 62 (if provided), as shown in FIG. 7. Even if a leukoreduction filter 62 is provided, the valve system may be controlled to cause the mixture to bypass the leukoreduction filter 62 and enter the red blood cell collection container 46 without being leukoreduced, as shown in FIG. 8. It is also within the scope of the present disclosure for the mixture to be routed through the leukoreduction filter 62 at the beginning of the collection stage, with the valve system being reconfigured during the collection stage to cause the mixture to bypass the leukoreduction filter 62, such that only a portion of the collected RBCs are leukoreduced.

In the configuration of FIG. 7 (in which the mixture is leukoreduced), valves 38a, 38b, and 38c are closed, while valve 38d is open, which directs the mixture from line L5 into line L11. The mixture flows through open valve 38d and the leukoreduction filter 62 and into line L12. The leukoreduced mixture then flows through open clamp 24b and into the red blood cell collection container 46.

In the configuration of FIG. 8 (in which the mixture is not leukoreduced), valves 38a, 38c, and 38d are closed, while valve 38b is open, which directs the mixture from line L5 into line L8 and then into line L13. The mixture flows through open valve 38b and into line L12, bypassing the leukoreduction filter 62. The non-leukoreduced mixture then flows through open clamp 24b and into the red blood cell collection container 46.

As described above, the mixture may be routed through the leukoreduction filter 62 at the beginning of the collection stage (as in FIG. 7), with the valve system being reconfigured during the collection stage to cause the mixture to bypass the leukoreduction filter 62 (as in FIG. 8), such that only a portion of the collected RBCs are leukoreduced. In one embodiment, pressure sensor 40b monitors the pressure of the leukoreduction filter 62. If the pressure sensor 40b detects that the pressure of the leukoreduction filter 62 has risen above a predetermined pressure threshold (which may be indicative of filter blockage), the controller may reconfigure the valve system (from the configuration of FIG. 7 to the configuration of FIG. 8) to cause the mixture to bypass the leukoreduction filter 62. The system may then alert the operator that the RBC product was not leukoreduced.

Regardless of whether the collected RBCs have been leukoreduced (or only partially leukoreduced), the collection stage continues until the target amount of whole blood has been drawn into the fluid flow circuit 12 from the blood source. In the case of a whole blood container 44 being used as a blood source (as in the illustrated embodiment) the collection stage will end when the whole blood container 44 (which may be filled with the target amount of whole blood) is empty, with different approaches possibly being employed to determine when the whole blood container 44 is empty. For example, in one embodiment, pressure sensor 40c monitors the hydrostatic pressure of the whole blood container 44. An empty whole blood container 44 may be detected when the hydrostatic pressure measured by pressure sensor 40c is at or below a threshold value. Alternatively (or additionally), the weight of the whole blood container 44 may be monitored by a weight scale, with an empty whole blood container 44 being detected when the weight is at or below a threshold value. In the case of a living donor (or in the event that the whole blood container 44 is provided with more than the target amount of blood), the volumetric flow rate of the whole blood pump 16 may be used to determine when the target amount of whole blood has been drawn into the fluid flow circuit 12.

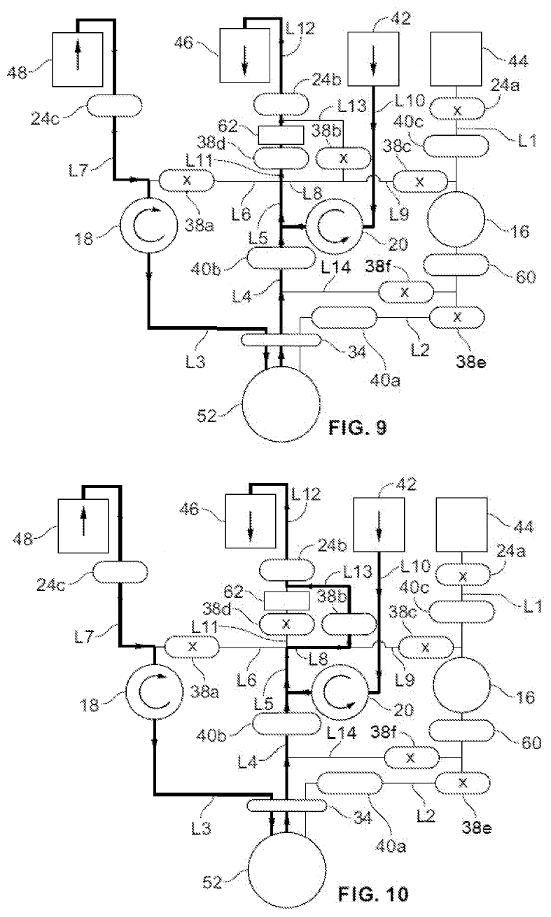

Once the target amount of whole blood has been drawn into the fluid flow circuit 12, the controller will transition the procedure to a “red blood cell recovery” stage, which is shown in FIG. 9. During the red blood cell recovery stage, air from the plasma collection container 48 (which was conveyed there during the blood prime stage) is used to recover the contents of the processing chamber 52 (which may be primarily RBCs) to reduce product loss.

In the illustrated embodiment, the whole blood pump 16 is deactivated, while the plasma pump 18 is operated in a reverse direction (with respect to its direction of operation up to this stage of the procedure). This draws the air from the plasma collection container 48 and into line L7. Valve 38a is closed, while clamp 24c is open, which directs the air through line L7, into and through line L3, and into the processing chamber 52 via the plasma outlet port. On account of the air flowing through the plasma outlet port, it will enter the processing chamber 52 at the low-g side. As additional air is introduced into the processing chamber 52, it will move from the low-g wall towards the high-g wall, thus displacing any liquid content through the red blood cell outlet port at the high-g side and into line L4. During this stage, the centrifuge 22 may be operated at a slower rate (e.g., in the range of approximately 1,000-2,000 rpm) to decrease the risk of an air blockage (as during the blood prime stage).

The additive pump 20 continues its operation, drawing additive solution from the additive solution container 42 and through line L10, to be mixed with the contents of the processing chamber 52 flowing through line L4 at the junction of the two lines L4 and L10. The mixture continues flowing into and through line L5. If the valve system was arranged in the configuration of FIG. 7 at the end of the collection stage (so as to direct flow through the leukoreduction filter 62), valves 38a, 38b, and 38c may remain closed, with valve 38d being open to direct the mixture into line L11 for leukoreduction, as in FIG. 9. On the other hand, if the valve system was arranged in the configuration of FIG. 8 at the end of the collection stage (so as to bypass the leukoreduction filter 62), valves 38a, 38c, and 38d may remain closed, with valve 38b being open to direct the mixture through lines L8 and L13 to bypass the leukoreduction filter 62, as in FIG. 10. As described above with regard to the collection stage, it is possible for the controller to change the configurations of the valve system from the configuration shown in FIG. 9 to the configuration of FIG. 10 during the red blood cell recovery stage to stop leukoreduction of the mixture (e.g., if the pressure of the leukoreduction filter 62 becomes too great).

Regardless of whether the mixture is filtered, it flows into line L12, through open clamp 24b, and into the red blood cell collection container 46. The red blood cell recovery stage continues until all of the air is removed from the plasma collection container 48. In one exemplary embodiment, the weight of the plasma collection container 48 may be monitored by a weight scale, with an empty plasma collection container 48 being detected when the weight is at or below a threshold value. Other approaches may also be employed to determine when to end the red blood cell recovery stage, such as using the optical sensor 34 to detect plasma flowing through line L3.

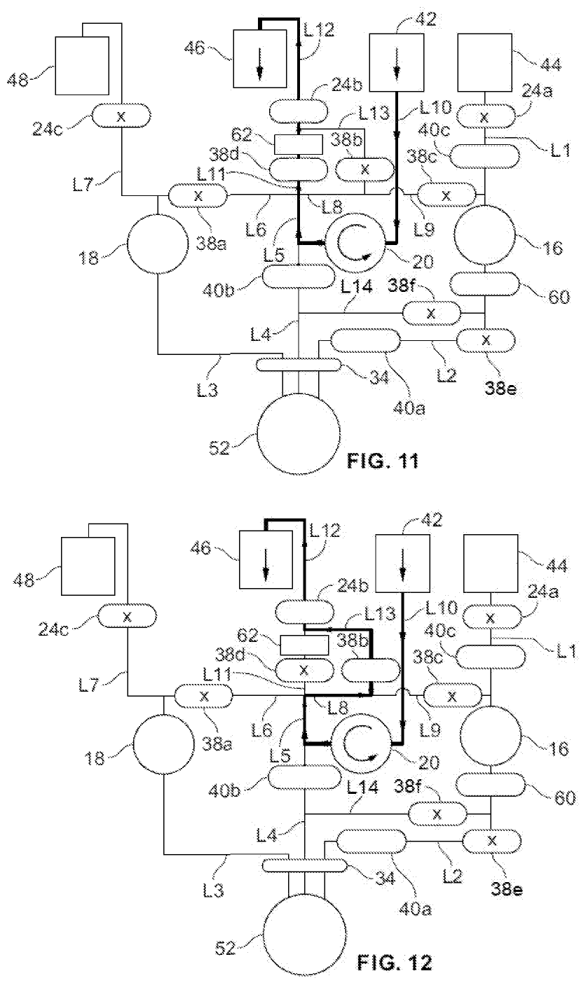

Once the red blood cell recovery stage is complete, the procedure will transition to an “additive solution flush” stage. During the additive solution flush stage, additive solution from the additive solution container 42 is conveyed into the red blood cell collection container 46 until a target amount of additive solution is in the red blood cell collection container 46. The only change in transitioning from the red blood cell recovery stage to the additive solution flush stage involves deactivating the plasma pump (and closing clamp 24c) to prevent plasma from being removed from the plasma collection container 48 (though it is also possible for the additive pump 20 to operate at a different rate). Thus, if the valve system was arranged to direct flow through the leukoreduction filter 62 at the end of the red blood cell recovery stage (as in FIG. 9), the additive solution flush stage will proceed as shown in FIG. 11. On the other hand, if the valve system was arranged to bypass the leukoreduction filter 62 at the end of the red blood cell recovery stage (as in FIG. 10), the additive solution flush stage will proceed as shown in FIG. 12. If the additive solution is pumped through the leukoreduction filter 62 during the additive solution flush stage (as in FIG. 11), the additive solution flowing through line L11 will flush residual RBCs in the leukoreduction filter 62 into the red blood cell collection container 46 (in addition to achieving a proper additive solution volume for the RBC product).

The additive solution flush stage will continue until a target amount of additive solution has been added to the red blood cell collection container 46. In one exemplary embodiment, the weight of the additive solution container 42 may be monitored by a weight scale, with a particular change in weight corresponding to the target amount of additive solution having been conveyed to the red blood cell collection container 46. Alternatively (or additionally), the weight of the red blood cell collection container 46 may be monitored by a weight scale, with a particular change in weight corresponding to the target amount of additive solution having been conveyed to the red blood cell collection container 46.

When the additive solution flush stage is complete, the system may proceed in one of two ways. If the RBCs are to remain in the (non-DEHP) red blood cell collection container 46, the system may transition to an “air evacuation” stage in which the red blood cell collection container 46 is “burped” to remove all residual air for storage (just as air was removed from the plasma collection container 48 during the red blood cell recovery stage). This is done by reversing the direction of operation of the additive pump 20, closing valve 38d (if not already closed at the end of the additive solution flush stage), and opening valve 38b (if not already open at the end of the additive solution flush stage). The additive pump 20 draws air out of the red blood cell collection container 46, through line L12 and open clamp 24b, into line L13 and through open valve 38b. The air continues through line L8, line L5, and line L10, with the air ending up in the additive solution container 42. Alternatively, rather than the air being evacuated from the red blood cell collection container 46 to the additive solution container 42, it is within the scope of the present disclosure for all or a portion of the air to be directed to a different location of the fluid flow circuit 12 (e.g., into the processing chamber 52 and/or into the whole blood container 44, if provided).

The air evacuation stage will continue until all of the air is removed from the red blood cell collection container 46, which may be determined (for example) by detecting a change in the weight of the red blood cell collection container 46 (e.g., using a weight scale).

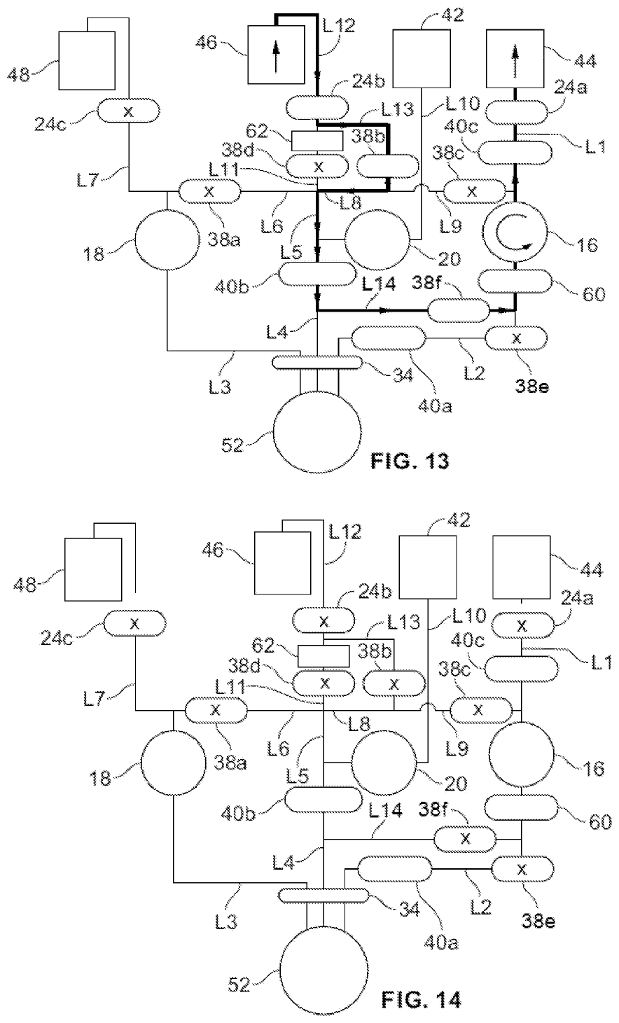

On the other hand, it is within the scope of the present disclosure for the RBCs to be stored in a container other than the red blood cell collection container 46 at the end of the procedure. In this case, rather than executing an air evacuation stage, the controller of the processing device 10 may instead execute a “red blood cell transfer” stage, as shown in FIG. 13. During the illustrated red blood cell transfer stage, the RBCs in the red blood cell collection container 46 are conveyed into the (non-DEHP) whole blood container 44 for collection and storage. This stage may be executed regardless of whether the red blood cell collection container 46 is formed of a DEHP-free material or a material including DEHP, though it may be especially advantageous when the red blood cell collection container 46 is formed of a material including DEHP.

As explained above, containers including an additive are incompatible with radiation sterilization. As the whole blood container 44 contains an anticoagulant solution, it cannot undergo radiation sterilization, such that the whole blood container must be provided separately from the remainder of the associated fluid flow circuit (which is sterilized via radiation) and separately sterilized (e.g., using steam). Once the whole blood container 44 has been sterilized, it is sterilely connected to one of the conduits of the fluid flow circuit, just prior to use of the fluid flow circuit.

As also explained above, a container formed of a material omitting DEHP (e.g., a citrate-plasticized material) is also incompatible with radiation sterilization and, thus, also requires sterile connection to the associated fluid flow circuit after sterilization. Accordingly, when a fluid flow circuit is provided with a non-DEHP red blood cell collection container and a whole blood container containing an anticoagulant solution, sterile connection of the two containers to the remainder of the fluid flow circuit is required, which increases the time and cost of any blood separation procedure. On the other hand, if the procedure were to end with the RBC product in the non-DEHP whole blood container 44, only one sterile connection (of the non-DEHP whole blood container 44) is required, as the red blood cell collection container 46 (which may be formed of a material including DEHP) may be pre-attached to the fluid flow circuit 12 and radiation sterilized therewith. As should be clear, this reduces the time and cost of executing a blood separation procedure.

During the red blood cell transfer stage, operation of the additive pump 20 is ceased, while the whole blood pump 16 is operated in a reverse direction (compared to its direction of operation during previous stages). Clamp 24a and valve 38f are also opened at this time. Valve 38d is closed (if not already closed at the end of the additive solution flush stage) and valve 38b is opened (if not already open at the end of the additive solution flush stage). The whole blood pump 16 draws the RBC product out of the red blood cell collection container 46, through line L12 and open clamp 24b, into line L13 and through open valve 38b. Valves 38a and 38c remain closed, such that the RBC product continues through line L8 and line L5. As the additive pump 20 is inoperative and valve 38e remains closed, the RBC product flows from line L5 into line L14 and through open valve 38f, with the RBC product finally flowing through line L1 (and open clamp 24a), ending up in the whole blood container 44.

The red blood cell transfer stage will continue until all of the RBC product (or at least a target amount) is removed from the red blood cell collection container 46, which may be determined (for example) by detecting a change in the weight of the red blood cell collection container 46 (e.g., using a weight scale). In one embodiment, the system state illustrated in FIG. 13 may continue after the red blood cell collection container 46 has been emptied of the RBC product in order to draw an amount of air from the red blood cell collection container 46. This may be considered to be an alternative “air evacuation” stage, as air is being removed from the red blood cell collection container 46 for a more complete transfer of the RBC product into the whole blood container 44, rather than for improved storage of the RBC product in the red blood cell collection container 46 (as in the above-described air evacuation stage). More particularly, during this alternative air evacuation stage, air from the red blood cell container 46 follows the same path through the fluid flow circuit 12 as the RBC product, thus flushing any residual RBC product from the path into the whole blood container 44 and ensuring that the RBC product is more completely collected.

Upon completion of the air evacuation stage or red blood cell transfer stage or alternative air evacuation stage, any of a number of post-processing stages may be executed. For example, FIG. 14 shows a “sealing” stage in which all of the clamps and valves are closed and all of the pumps are deactivated. In the illustrated embodiment (in which the RBC product is stored in the whole blood container 44), the line L1 connected to the whole blood container 44 and the line L7 connected to the plasma collection container 48 are sealed and optionally severed for storage of the RBC and plasma products. Alternatively, if the RBC product is to be stored in the red blood cell collection container 46, the line L12 connected to the red blood cell collection container 46 (rather than the line L1 connected to the whole blood container 44) may be sealed and optionally severed for the storage of the RBC product.

For any lines that are severed, the associated containers may be stored, while the remainder of the fluid flow circuit 12 is disposed of. The lines may be sealed (and optionally severed) according to any suitable approach, which may include being sealed by RF sealers incorporated or associated with the appropriate clamps, for example. In another embodiment, the fluid flow circuit 12 may be removed from the processing device 10, with the appropriate lines being sealed (and optionally severed) using a dedicated sealing device.

### Aspects

Aspect 1. A blood processing system, comprising: a reusable processing device including a pump system, a blood separation assembly, and a controller configured to execute a blood processing procedure; and a disposable fluid flow circuit including a processing chamber received by the blood separation assembly, a red blood cell collection container containing an additive solution, an additive solution container, and a plurality of conduits fluidly connecting the components of the fluid flow circuit, wherein the controller is configured to actuate the pump system to convey the additive solution from the red blood cell collection container into the additive solution container, actuate the pump system to convey blood from a blood source into the processing chamber, actuate the blood separation assembly to separate red blood cells from the blood in the processing chamber, actuate the pump system to convey at least a portion of the separated red blood cells out of the processing chamber and into the red blood cell collection container, and actuate the pump system to convey at least a portion of the additive solution from the additive solution container into the red blood cell collection container.

Aspect 2. The blood processing system of Aspect 1, wherein the additive solution container is substantially empty prior to the controller actuating the pump system to convey the additive solution from the red blood cell collection container into the additive solution container.

Aspect 3. The blood processing system of any one of the preceding Aspects, wherein the red blood cell collection container is formed of a material omitting di-2-ethylhexyl phthalate.

Aspect 4. The blood processing system of any one of the preceding Aspects, wherein the additive solution container is formed of a material including di-2-ethylhexyl phthalate. Aspect 5. The blood processing system of any one of the preceding

Aspects, wherein the red blood cell collection container is formed of a material including a citrate plasticizer.

Aspect 6. The blood processing system of any one of the preceding Aspects, wherein the red blood cell collection container is separately provided from the fluid flow circuit and configured to be sterilely connected to one of the conduits of the fluid flow circuit.

Aspect 7. The blood processing system of any one of the preceding Aspects, wherein the additive solution container is pre-attached to one of the conduits of the fluid flow circuit.

Aspect 8. The blood processing system of any one of the preceding Aspects, wherein the controller is configured to execute the blood processing procedure so as to retain said at least a portion of the separated red blood cells and said at least a portion of the additive solution in the red blood cell collection container at the end of the blood processing procedure.

Aspect 9. The blood processing system of any one of Aspects 1-7, wherein the controller is configured to execute the blood processing procedure so as to retain said at least a portion of the separated red blood cells and said at least a portion of the additive solution in a container other than the red blood cell collection container at the end of the blood processing procedure.

Aspect 10. The blood processing system of Aspect 9, wherein the blood source comprises a whole blood container, and the controller is configured to execute the blood processing procedure so as to retain said at least a portion of the separated red blood cells and said at least a portion of the additive solution in the whole blood container at the end of the blood processing procedure.

Aspect 11. A method for separating red blood cells from whole blood, comprising: conveying an additive solution from a red blood cell collection container into an additive solution container; conveying blood from a blood source into a processing chamber; separating red blood cells from the blood in the processing chamber; conveying at least a portion of the separated red blood cells out of the processing chamber and into the red blood cell collection container; and conveying at least a portion of the additive solution from the additive solution container into the red blood cell collection container.

Aspect 12. The method of Aspect 11, wherein the additive solution container is substantially empty prior to conveying the additive solution from the red blood cell collection container into the additive solution container.

Aspect 13. The method of any one of Aspects 11-12, wherein the red blood cell collection container is formed of a material omitting di-2-ethylhexyl phthalate.

Aspect 14. The method of any one of Aspects 11-13, wherein the additive solution container is formed of a material including di-2-ethylhexyl phthalate.

Aspect 15. The method of any one of Aspects 11-14, wherein the red blood cell collection container is formed of a material including a citrate plasticizer.

Aspect 16. The method of any one of Aspects 11-15, further comprising sterilely connecting the red blood cell collection container to a fluid flow circuit including the processing chamber.

Aspect 17. The method of any one of Aspects 11-16, wherein the additive solution container is pre-attached to a fluid flow circuit including the processing chamber.

Aspect 18. The method of any one of Aspects 11-17, wherein the method concludes with said at least a portion of the separated red blood cells and said at least a portion of the additive solution in the red blood cell collection container.

Aspect 19. The method of any one of Aspects 11-17, wherein the method concludes with said at least a portion of the separated red blood cells and said at least a portion of the additive solution in a container other than the red blood cell collection container.

Aspect 20. The method of Aspect 19, wherein the blood source comprises a whole blood container, and the method concludes with said at least a portion of the separated red blood cells and said at least a portion of the additive solution in the whole blood container.

Aspect 21. A blood processing system, comprising: a reusable processing device including a pump system, a blood separation assembly, and a controller configured to execute a blood processing procedure; and a disposable fluid flow circuit including a processing chamber received by the blood separation assembly, a red blood cell collection container, a whole blood container, and a plurality of conduits fluidly connecting the components of the fluid flow circuit, wherein the controller is configured to actuate the pump system to convey blood from the whole blood container into the processing chamber, actuate the blood separation assembly to separate red blood cells from the blood in the processing chamber, actuate the pump system to convey at least a portion of the separated red blood cells out of the processing chamber and into the red blood cell collection container, and actuate the pump system to convey said at least a portion of the separated red blood cells from the red blood cell collection container into the whole blood container.

Aspect 22. The blood processing system of Aspect 21, wherein the red blood cell collection container is formed of a material including di-2-ethylhexyl phthalate.

Aspect 23. The blood processing system of Aspect 21, wherein the red blood cell collection container is formed of a material omitting di-2-ethylhexyl phthalate.

Aspect 24. The blood processing system of any one of Aspects 21-23, wherein the whole blood container is formed of a material omitting di-2-ethylhexyl phthalate.

Aspect 25. The blood processing system of any one of Aspects 21-24, wherein the blood separation assembly is configured as a centrifuge.

Aspect 26. The blood processing system of any one of Aspects 21-25, wherein the controller is further configured to actuate the pump system to convey an amount of air from the red blood cell collection container after conveying said at least a portion of the separated red blood cells from the red blood cell collection into the whole blood container.

Aspect 27. The blood processing system of any one of Aspects 21-26, wherein the fluid flow circuit includes an additive solution container, and the controller is configured to actuate the pump system to convey an additive solution from the additive solution container into the red blood cell collection container prior to actuating the pump system to convey said at least a portion of the separated red blood cells from the red blood cell collection container into the whole blood container.

Aspect 28. The blood processing system of Aspect 27, wherein the additive solution container is substantially empty prior to the controller actuating the pump system to convey blood from the whole blood container into the processing chamber.

Aspect 29. The blood processing system of any one of Aspects 27-28,wherein the red blood cell collection container initially includes the additive solution, and the controller is configured to actuate the pump system to convey the additive solution from the red blood cell collection container into the additive solution container prior to actuating the pump system to convey blood from the whole blood container into the processing chamber.

Aspect 30. The blood processing system of any one of Aspects 27-29,wherein the additive solution container is formed of a material including di-2-ethylhexyl phthalate.

Aspect 31. A method for separating red blood cells from whole blood, comprising: conveying blood from a whole blood container into a processing chamber; separating red blood cells from the blood in the processing chamber; conveying at least a portion of the separated red blood cells out of the processing chamber and into a red blood cell collection container; and conveying said at least a portion of the separated red blood cells out of the red blood cell collection container and into the whole blood container.

Aspect 32. The method of Aspect 31, wherein the red blood cell collection container is formed of a material including di-2-ethylhexyl phthalate.

Aspect 33. The method of Aspect 31, wherein the red blood cell collection container is formed of a material omitting di-2-ethylhexyl phthalate.

Aspect 34. The method of any one of Aspects 31-33, wherein the whole blood container is formed of a material omitting di-2-ethylhexyl phthalate.

Aspect 35. The method of any one of Aspects 31-34, wherein said separating red blood cells from the blood in the processing chamber includes separating the red blood cells from the blood via centrifugation.

Aspect 36. The method of any one of Aspects 31-35, further comprising conveying an amount of air from the red blood cell collection container after conveying said at least a portion of the separated red blood cells from the red blood cell collection into the whole blood container.

Aspect 37. The method of any one of Aspects 31-36, further comprising conveying an additive solution from an additive solution container into the red blood cell collection container prior to conveying said at least a portion of the separated red blood cells from the red blood cell collection container into the whole blood container.

Aspect 38. The method of Aspect 37, wherein the additive solution container is substantially empty prior to conveying the blood from the whole blood container into the processing chamber.

Aspect 39. The method of any one of Aspects 37-38, wherein the red blood cell collection container initially includes the additive solution, and the method includes conveying the additive solution from the red blood cell collection container into the additive solution container prior to conveying the blood from the whole blood container into the processing chamber.

Aspect 40. The method of any one of Aspects 37-39, wherein the additive solution container is formed of a material including di-2-ethylhexyl phthalate.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A blood processing system, comprising:
a reusable processing device including
a pump system,
a blood separation assembly, and
a controller configured to execute a blood processing procedure; and
a disposable fluid flow circuit including
a processing chamber received by the blood separation assembly,
a red blood cell collection container containing an additive solution,
an additive solution container, and
a plurality of conduits fluidly connecting the components of the fluid flow circuit, wherein the controller is configured to
actuate the pump system to convey the additive solution from the red blood cell collection container into the additive solution container,
actuate the pump system to convey blood from a blood source into the processing chamber,
actuate the blood separation assembly to separate red blood cells from the blood in the processing chamber,
actuate the pump system to convey at least a portion of the separated red blood cells out of the processing chamber and into the red blood cell collection container, and
actuate the pump system to convey at least a portion of the additive solution from the additive solution container into the red blood cell collection container.

2. The blood processing system of claim 1, wherein the additive solution container is substantially empty prior to the controller actuating the pump system to convey the additive solution from the red blood cell collection container into the additive solution container.

3. The blood processing system of claim 1, wherein the red blood cell collection container is formed of a material omitting di-2-ethylhexyl phthalate and/or including a citrate plasticizer and the additive solution container is formed of a material including di-2-ethylhexyl phthalate.

4. (canceled)

5. (canceled)

6. (canceled)

7. (canceled)

8. The blood processing system of claim 1, wherein the controller is configured to execute the blood processing procedure so as to retain said at least a portion of the separated red blood cells and said at least a portion of the additive solution in the red blood cell collection container or in a container other than the red blood cell collection container at the end of the blood processing procedure.

9. (canceled)

10. The blood processing system of claim 1, wherein
the blood source comprises a whole blood container, and
the controller is configured to execute the blood processing procedure so as to retain said at least a portion of the separated red blood cells and said at least a portion of the additive solution in the whole blood container at the end of the blood processing procedure.

11. A method for separating red blood cells from whole blood, comprising:
conveying an additive solution from a red blood cell collection container into an additive solution container;
conveying blood from a blood source into a processing chamber;
separating red blood cells from the blood in the processing chamber;
conveying at least a portion of the separated red blood cells out of the processing chamber and into the red blood cell collection container; and
conveying at least a portion of the additive solution from the additive solution container into the red blood cell collection container.

12. The method of claim 11, wherein the additive solution container is substantially empty prior to conveying the additive solution from the red blood cell collection container into the additive solution container.

13. The method of claim 11, wherein the red blood cell collection container is formed of a material omitting di-2-ethylhexyl phthalate and/or including a citrate plasticizer and the additive solution container is formed of a material including di-2-ethylhexyl phthalate.

14. (canceled)

15. (canceled)

16. (canceled)

17. (canceled)

18. The method of claim 11, wherein the method concludes with said at least a portion of the separated red blood cells and said at least a portion of the additive solution in the red blood cell collection container or in a container other than the red blood cell solution container.

19. (canceled)

20. The method of claim 11, wherein
the blood source comprises a whole blood container, and
the method concludes with said at least a portion of the separated red blood cells and said at least a portion of the additive solution in the whole blood container.

21. A blood processing system, comprising:
a reusable processing device including
a pump system,
a blood separation assembly, and
a controller configured to execute a blood processing procedure; and
a disposable fluid flow circuit including
a processing chamber received by the blood separation assembly,
a red blood cell collection container,
a whole blood container, and
a plurality of conduits fluidly connecting the components of the fluid flow circuit, wherein the controller is configured to
actuate the pump system to convey blood from the whole blood container into the processing chamber,
actuate the blood separation assembly to separate red blood cells from the blood in the processing chamber,
actuate the pump system to convey at least a portion of the separated red blood cells out of the processing chamber and into the red blood cell collection container, and
actuate the pump system to convey said at least a portion of the separated red blood cells from the red blood cell collection container into the whole blood container.

22. The blood processing system of claim 21, wherein the red blood cell collection container is formed of a material including di-2-ethylhexyl phthalate.

23. The blood processing system of claim 21, wherein the red blood cell collection container and/or the whole blood container is formed of a material omitting di-2-ethylhexyl phthalate.

24. (canceled)

25. (canceled)

26. The blood processing system of claim 21, wherein the controller is further configured to actuate the pump system to convey an amount of air from the red blood cell collection container after conveying said at least a portion of the separated red blood cells from the red blood cell collection into the whole blood container.

27. The blood processing system of claim 21, wherein
the fluid flow circuit includes an additive solution container and the red blood cell collection container initially includes an additive solution, and
the controller is configured to actuate the pump system to convey the additive solution from the red blood cell collection container into the additive solution container prior to actuating the pump system to convey blood from the whole blood container into the processing chamber, and wherein the controller is configured to actuate the pump system to convey the additive solution from the additive solution container into the red blood cell collection container prior to actuating the pump system to convey said at least a portion of the separated red blood cells from the red blood cell collection container into the whole blood container.

28. (canceled)

29. (canceled)

30. (canceled)

31. A method for separating red blood cells from whole blood, comprising:
conveying blood from a whole blood container into a processing chamber;
separating red blood cells from the blood in the processing chamber;
conveying at least a portion of the separated red blood cells out of the processing chamber and into a red blood cell collection container; and
conveying said at least a portion of the separated red blood cells out of the red blood cell collection container and into the whole blood container.

32. The method of claim 31, wherein the red blood cell collection container is formed of a material including di-2-ethylhexyl phthalate.

33. The method of claim 31, wherein the red blood cell collection container and/or the whole blood container is formed of a material omitting di-2-ethylhexyl phthalate.

34. (canceled)

35. (canceled)

36. The method of claim 31, further comprising conveying an amount of air from the red blood cell collection container after conveying said at least a portion of the separated red blood cells from the red blood cell collection into the whole blood container.

37. The method of claim 31, wherein the red blood cell collection container initially includes an additive solution and further comprising conveying the additive solution from the red blood cell collection container into the additive solution container prior to conveying the blood form the whole blood container into the processing chamber, and conveying an additive solution from the additive solution container into the red blood cell collection container prior to conveying said at least a portion of the separated red blood cells from the red blood cell collection container into the whole blood container.

38. (canceled)

39. (canceled)

40. (canceled)
